(19) United States
(12) Patent Application Publication
(10) Pub. No.: US 2018/0070829 A1
(43) Pub. Date: Mar. 15, 2018

(54) OPTICAL SENSOR MODULE, BIOLOGICAL INFORMATION DETECTING APPARATUS, AND ELECTRONIC INSTRUMENT

(71) Applicant: SEIKO EPSON CORPORATION, Tokyo (JP)

(72) Inventors: Takanori IWAWAKI, Chino-shi (JP); Akira UEMATSU, Suwa-shi (JP); Atsushi MATSUO, Azumino-shi (JP)

(73) Assignee: SEIKO EPSON CORPORATION, Tokyo (JP)

(21) Appl. No.: 15/695,233

(22) Filed: Sep. 5, 2017

(30) Foreign Application Priority Data
Sep. 12, 2016 (JP) 2016-177377

(51) Int. Cl. A61B 5/00; A61B 5/024; A61B 5/0432; H01L 31/0216

(52) U.S. Cl. CPC A61B 5/0059; A61B 5/6801; H01L 31/02162; A61B 5/681; A61B 5/04325; A61B 5/02427

(57) ABSTRACT

An optical sensor module includes a light emitter that radiates light to a target object, a light receiver that receives light from the target object, a deformable substrate on which the light emitter and the light receiver are provided, and a reinforcing plate that reinforces the strength of the substrate.

### CROSS-REFERENCES TO RELATED APPLICATIONS

This application claims priority to Japanese Patent Application No. 2016-177377, filed Sep. 12, 2016, the entirety of which is herein incorporated by reference.

### BACKGROUND

### 1. Technical Field

The present invention relates to an optical sensor module, a biological information detecting apparatus, an electronic instrument, and the like.

### 2. Related Art

There is a widely known optical sensor (photoelectric sensor) of related art including a light emitter and a light receiver. As the optical sensor, for example, a pulse wave sensor for measuring a pulse wave is widely known. A pulse wave sensor is so configured that the light emitter radiates light toward a subject (skin surface) and the light receiver receives the light having been reflected of f or having passed through the subject (interior of human body). For example, a reflection-type pulse wave sensor is so configured that the light emitter and the light receiver are arranged side by side and a light transmissive member is provided above the light emitter and the light receiver. When the pulse wave sensor is used (pulse wave is measured), the light transmissive member comes into intimate contact with the surface of the skin of a finger or an arm of the human body.

JP-A-2007-175415 discloses an optical sensor in which a light emitter and a light receiver are mounted on a substrate in which a predetermined wiring pattern is formed.

In JP-A-2007-175415, a solder film is used to bond the light emitter and the light receiver onto the substrate. It is therefore believed that cost reduction is achieved and volume production is also readily performed, as compared with an approach to a three-dimensional arrangement (for example, approach disclosed in JP-A-2007-175415 with reference to FIGS. 16 to 19). In the approach disclosed in JP-A-2007-175415, however, the thickness of the optical sensor module is determined by the sum of the thickness of the substrate and the thickness of parts mounted on the substrate. In the approach disclosed in JP-A-2007-175415, to suppress noise resulting from swing motion of the light emitter and the light receiver, a substrate having adequate strength is required. In this case, the substrate inevitably has a thickness to some extent, and it is therefore difficult to reduce the thickness of the optical sensor module. Further, in a configuration in which a cable is used to connect the optical sensor module to a main substrate in a wearable apparatus, since a connection portion where the cable is connected to each of the substrates requires a physical space, it is not easy to reduce the size of the wearable apparatus in some cases.

### SUMMARY

An advantage of some aspects of the invention is to provide an optical sensor module, a biological information detecting apparatus, and an electronic instrument that have small thickness and sufficient strength.

An aspect of the invention relates to an optical sensor module including a light emitter that radiates light to a target object, a light receiver that receives light from the target object, a deformable substrate on which the light emitter and the light receiver are provided, and a reinforcing plate that reinforces strength of the substrate.

In the aspect of the invention, a deformable substrate is used as the substrate on which the light emitter and the light receiver are provided, and the reinforcing plate is used to reinforce the strength of the substrate. As a result, the thickness of the optical sensor module can be reduced, and the strength thereof can be ensured. Further, since no contact point between the optical sensor module and a flexible cable is required, space saving is achieved.

In the aspect of the invention, part of the reinforcing plate may form a light blocker that blocks direct light from the light emitter to the light receiver.

According to the configuration described above, the number of parts can be reduced, and the optical sensor module can be efficiently configured.

In the aspect of the invention, the optical sensor module may further include a light blocker that is formed as a member separate from the reinforcing plate and blocks direct light from the light emitter to the light receiver.

According to the configuration described above, the reinforcing plate and the light blocker can be members separate from each other, whereby the shape of each of the members can be simplified and other advantages can be provided.

In the aspect of the invention, the optical module sensor may further include a connection portion that connects the substrate and the reinforcing plate to each other.

According to the configuration described above, the substrate and the reinforcing plate can be appropriately connected to each other.

In the aspect of the invention, the connection portion may connect the substrate and the reinforcing plate to each other with solder.

According to the configuration described above, the substrate and the reinforcing plate can be connected to each other with solder.

In the aspect of the invention, in a plan view viewed from a side facing the target object, a plurality of connection portions each of which is formed of the connection portion may be so provided as to surround the light emitter and the light receiver.

According to the configuration described above, deformation of the substrate around the light emitter and the light receiver can be suppressed, whereby the detection accuracy can be improved and other advantages can be provided.

In the aspect of the invention, the connection portion may be disposed in a region along a first edge of the substrate and a region along a second edge of the substrate that faces the first edge.

According to the configuration described above, the substrate and the reinforcing plate can be appropriately connected to each other, and deformation of the substrate can be efficiently suppressed.

In the aspect of the invention, in a plan view viewed from a side facing the target object, the reinforcing plate may be so provided as to contain the light emitter and the light receiver.

According to the configuration described above, deformation of the substrate around the light emitter and the light receiver can be suppressed, whereby the detection accuracy can be improved and other advantages can be provided.

In the aspect of the invention, in a plan view viewed from a side facing the target object, the reinforcing plate may have at least one hole section that exposes the light emitter and the light receiver.

According to the configuration described above, the light emitter and the light receiver can be exposed when the reinforcing plate is connected to the substrate.

In the aspect of the invention, the reinforcing plate may have, as the at least one hole section, a first hole section that exposes the light emitter and a second hole section that exposes the light receiver.

According to the configuration described above, the light emitter and the light receiver can be each individually provided with a hole section for exposure.

In the aspect of the invention, in a plan view viewed from a side facing the target object, the reinforcing plate may have a first hole section that exposes the light emitter and a second hole section that exposes the light receiver, and the light blocker may be provided at least in a position between the first hole section and the second hole section.

According to the configuration described above, direct light from light emitter to the light receiver can be efficiently blocked.

In the aspect of the invention, the optical sensor module may further include a detector at least including an amplification section that amplifies a detection signal from the light receiver, and the detector may be provided on the substrate.

According to the configuration described above, the detector can be mounted on the substrate.

In the aspect of the invention, the substrate may be provided with a connector section electrically connected to a second substrate provided with a processing section that carries out a process based on the detection signal from the light receiver.

According to the configuration described above, a signal based on a result of the light received by the light receiver can be outputted to another substrate.

In the aspect of the invention, L1<L2 and L1<L3 may be satisfied, where L1 represents a distance from the connector section to the detector, L2 represents a distance from the connector section to the light emitter, and L3 represents a distance from the connector section to the light receiver.

According to the configuration described above, the light emitter, the light receiver, and the detector can be appropriately disposed on the substrate and other advantages can be provided.

In the aspect of the invention, the reinforcing plate may be formed of a metal member or a resin member.

According to the configuration described above, a reinforcing plate formed of a metal member or a resin member can be used.

In the aspect of the invention, the deformable substrate may be a flexible printed circuit.

According to the configuration described above, a very thin flexible printed circuit can be used as the substrate.

Another aspect of the invention relates to a biological information detecting apparatus including any of the optical sensor module described above.

Another aspect of the invention relates to an electronic instrument including any of the optical sensor module described above.

### BRIEF DESCRIPTION OF THE DRAWINGS

The invention will be described with reference to the accompanying drawings, wherein like numbers reference like elements.

FIG. 1 is a side view of an optical sensor module based on a related art approach.

FIG. 2 is a side view of an optical sensor module according to an embodiment of the invention.

FIG. 3 is a plan view of a substrate in a first embodiment.

FIG. 4 is a plan view of the substrate and a reinforcing plate in the first embodiment.

FIG. 5 is a plan view of the reinforcing plate in the first embodiment.

FIG. 6 is a perspective view of the reinforcing plate shown in the first embodiment.

FIG. 7 is a development of the reinforcing plate in the first embodiment.

FIG. 8 is a plan view of the optical sensor module according to the first embodiment.

FIG. 9 is a circuit diagram of the optical sensor module.

FIG. 10 is a plan view of a substrate in a second embodiment.

FIG. 11 is a plan view of a reinforcing plate in the second embodiment.

FIG. 12 is a perspective view of the reinforcing plate in the second embodiment.

FIG. 13 is a plan view of an optical sensor module according to the second embodiment.

FIG. 14 is a side view of the optical sensor module according to the second embodiment.

FIG. 15 is a plan view of a light blocker in a third embodiment.

FIG. 16 is a perspective view of the light blocker in the third embodiment.

FIG. 17 is a plan view of a reinforcing plate in the third embodiment.

FIG. 18 is a plan view of an optical sensor module according to the third embodiment.

FIG. 19 is a side view of the optical sensor module according to the third embodiment.

FIG. 20 is an exploded view of a biological information detecting apparatus.

FIG. 21 shows an exterior appearance of the biological information detecting apparatus.

FIG. 22 shows an exterior appearance of the biological information detecting apparatus.

FIG. 23 is a perspective view of key parts of a printing apparatus.

### DESCRIPTION OF EXEMPLARY EMBODIMENTS

An embodiment of the present invention will be described below. It is not intended that the present embodiment described below unduly limits the contents of the invention set forth in the appended claims. Further, all configurations described in the present embodiment are not necessarily essential configuration requirements of the invention.

### 1. Approach in Present Embodiment

An approach in the present embodiment will first be described. There are a known optical sensor module of related art including a light emitter and a light receiver and a variety of known apparatus of related art each including the optical sensor module. For example, an optical sensor module is used in a biological information detecting apparatus that acquires biological information by irradiating a subject (living body) with light from the light emitter and receiving the light reflected off the living body with the light receiver. In the optical sensor module accommodated in the biological information detecting apparatus, the light emitter radiates light that belongs to a wavelength band that is likely to be absorbed by blood (hemoglobin contained in blood in narrow sense). In a case where the amount of blood flow is large and the amount of hemoglobin is therefore large, the amount of absorbed light is large and the intensity of the reflected light is small. Conversely, in a case where the amount of blood flow is small and the amount of hemoglobin is therefore small, the amount of absorbed light is small and the intensity of the reflected light is large. In this case, since a change in a signal from the light receiver (AC component) represents a change in the amount of blood flow, the biological information detecting apparatus can determine pulse wave information on the basis of the signal from the light receiver.

The light emitter may instead be configured to radiate light that belongs to a first wavelength band where oxygenated hemoglobin absorption coefficient is relatively large and light that belongs to a second wavelength band where reduced hemoglobin absorption coefficient is relatively large. In this case, a reception signal representing reflected light resulting from the light that belongs to the first wavelength band and a reception signal representing reflected light resulting from the light that belongs to the second wavelength band can be used to estimate the ratio between the oxygenated hemoglobin and the reduced hemoglobin in the blood. That is, the biological information detection apparatus can determine oxygen saturation (arterial oxygen saturation SpO₂ in narrow sense) in the blood as the biological information on the basis of the signal from the light receiver.

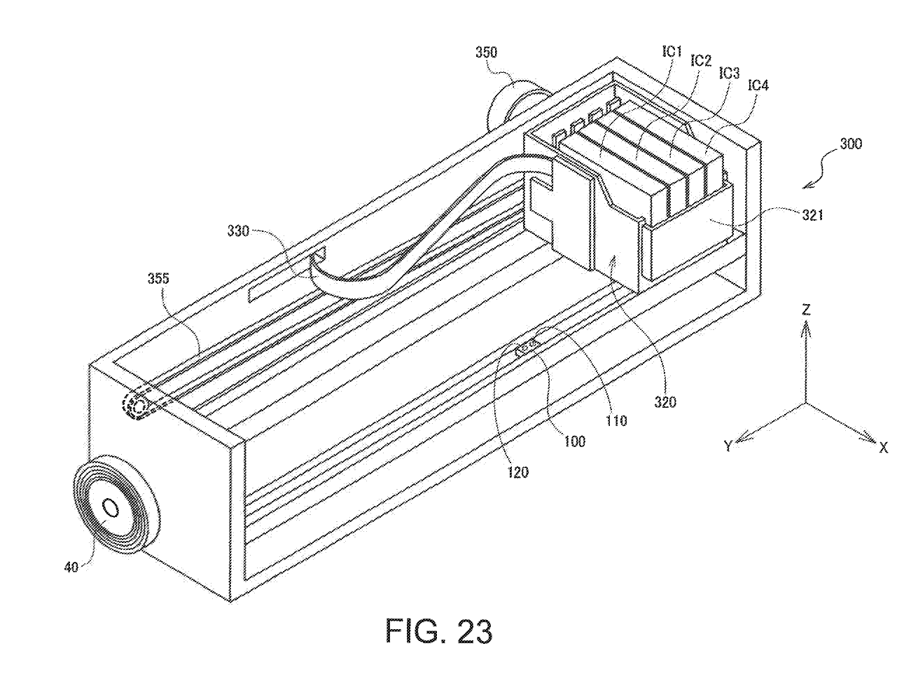

The information detected by an optical sensor module including a light emitter and a light receiver is not limited to biological information. For example, in a case of a printing apparatus (liquid consuming apparatus) that will be described later with reference to FIG. 23, the difference in refractive index between a liquid (ink), which is a consumed object, and the air is used to detect whether or not the liquid is present (amount of remaining liquid). Further, the distance from an optical sensor module to a target object can be measured. A known example of distance measurement using an optical sensor module is a time-of-flight method for measuring the period from the point of time when a light emitter radiates light to the point of time when a light receiver receives the light reflected off a target object.

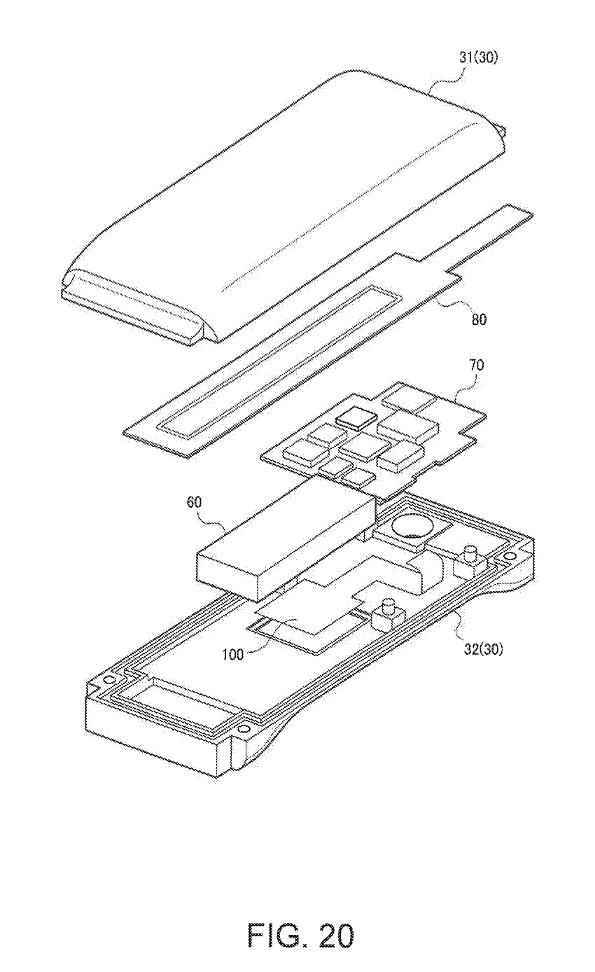

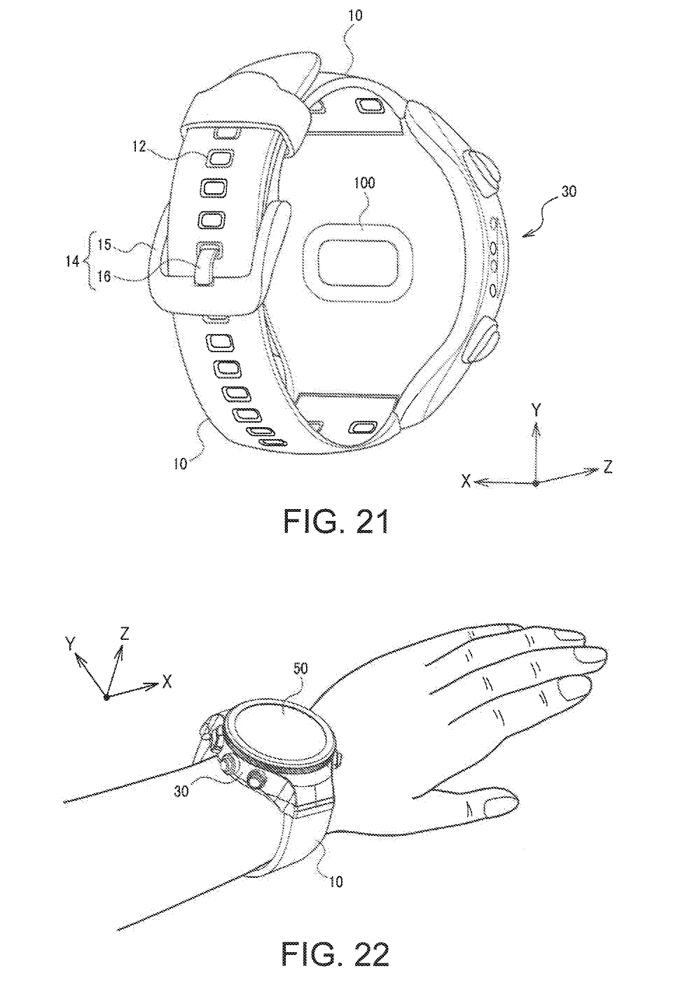

An optical sensor module conceivably used in a variety of apparatus as described above is strongly required to be small in thickness. The reason for this is that reduction in thickness of an optical sensor module allows reduction in thickness and size of an apparatus including the optical sensor module. For example, as will be described later with reference to FIGS. 21 and 22 and other figures, a biological information detecting apparatus 200 including an optical sensor module 100 is conceivably a wearable apparatus worn by a user. In this case, when the biological information detecting apparatus 200 is large, discomfort from the worn biological information detecting apparatus increases, and it is therefore very important to reduce the size of the apparatus. The biological information detecting apparatus 200 is also provided with a battery 60, a second substrate 70, on which a processing section (such as DSP) is mounted, an OLED panel 80, and other parts other than the optical sensor module 100, as will be described later with reference to FIG. 20. That is, to reduce the size of the biological information detecting apparatus 200, it is important to reduce the size of each part, and the optical sensor module 100 is no exception. Further, even in other electronic instruments, such as a liquid consuming apparatus and a distance measuring apparatus, it is not guaranteed that there is enough room for arrangement of the optical sensor module 100, and reduction in thickness of the optical sensor module 100 is similarly greatly advantageous.

In contrast, it is conceivable to employ, for example, an approach to providing a substrate with a groove (recess) or a hole and burying the light emitter, the light receiver, and other mounted parts in the groove or the hole. The thickness can therefore be reduced by the amount corresponding to the depth of the groove or the hole. The mounting method described above, however, increases the cost and provides low productivity. In consideration of this point, JP-A-2007-175415 discloses an approach to two-dimensional arrangement of the light emitter and the light receiver. In the approach disclosed in JP-A-2007-175415, however, no consideration is given to reduction in the thickness of the optical sensor module.

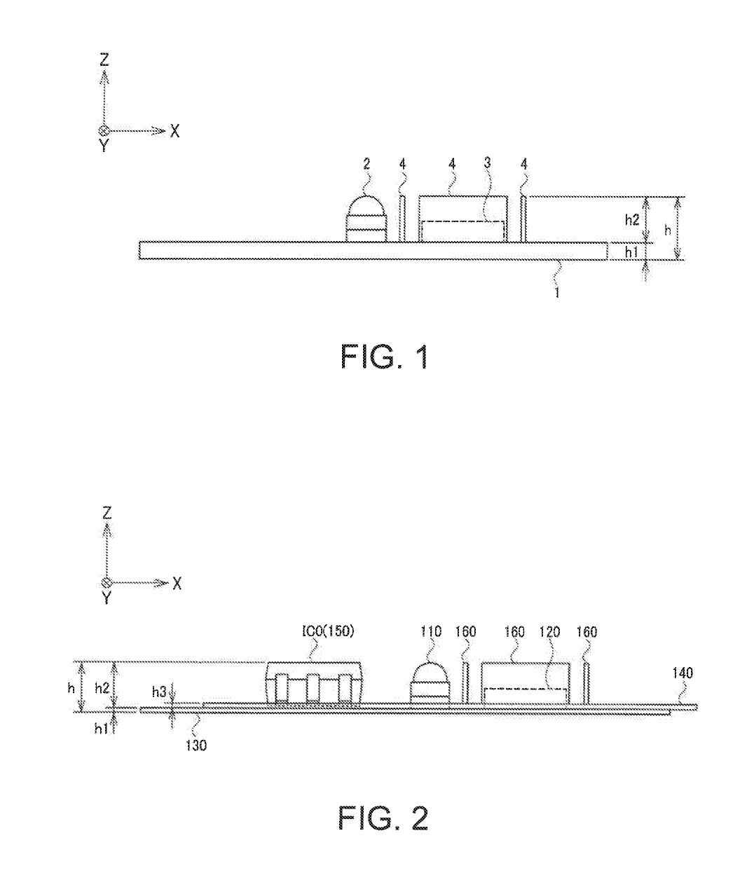

In an optical sensor module, since deformation (bend and deflection) of a substrate causes noise to occur, it is usual to use a substrate having strength to some degree, and such a substrate is thick to some degree. For example, a solid silicon substrate has a thickness of about 500 μm. FIG. 1 describes a related art approach disclosed, for example, in JP-A-2007-175415 and is a side view in a case where a substrate 1 has strength to some degree but has no groove or hole provided therein and a light emitter 2, a light receiver 3, and a light blocker 4 are mounted on the substrate 1 (side view of laterally viewed mounting surface). In the related art approach, the thickness h of the optical sensor module corresponds to the sum of the thickness h1 of the substrate and the thickness h2 of the parts mounted on the substrate, as shown in FIG. 1. To reduce the thickness of the optical sensor module, it is necessary to reduce h1 or h2. However, since the size of each part itself has been determined to some degree, it is not easy to reduce h2. Further, in a case where the intensity of outputted light is required to be large, an LED (light emitting diode) having a large lens is used as the light emitter 2 in some cases, and it is difficult to reduce the thickness of each part from the viewpoint of required performance and other factors in some cases. That is, to reduce the thickness of an optical sensor module, an approach to reduction in thickness of the substrate is considered effective.

When the degree of deformation of the substrate increases, however, the positional relationship between the light emitter and a target object, the positional relationship between the light receiver and the target object, the positional relationship between the light emitter and the light receiver, and other factors change, and a reception signal from the light receiver therefore changes. In this case, whether a change in the reception signal results from a change in the target object to be detected or the deformation of the substrate cannot be determined, resulting in a decrease in detection accuracy. The change in the target object to be detected is a change in the amount of blood flow due to the beats in the case of the biological information detecting apparatus 200 described above. That is, it is assumed in the case described above that the substrate used in the optical sensor module has strength large enough not to experience excessive deformation. Therefore, simply replacing the substrate in the related art approach, such as JP-A-2007-175415, with a thinner substrate leaves an accuracy problem.

In view of the fact described above, what is proposed in the present embodiment is an optical sensor module 100 on which parts can be readily mounted and which has strength to some degree and allows reduction in thickness. The optical sensor module 100 according to the present embodiment includes a light emitter 110, which irradiates a target object with light, a light receiver 120, which receives light from the target object, a deformable substrate 130, on which the light emitter 110 and the light receiver 120 are mounted, and a reinforcing plate 140, which reinforces the strength of the substrate. The light emitter 110 is, for example, an LED, and the light receiver 120 is, for example, a PD (photodiode), but not necessarily. One light emitter 110 may be provided, or a plurality of light emitters 110 may be provided, as seen from the example of arterial oxygen saturation described above. Similarly, one light receiver 120 may be provided, or a plurality of light receivers 120 may be provided. In the case where a plurality of light emitters 110 and light receivers 120 are provided, a plurality of light emitters 110 and light receivers 120 having the same optical characteristics (wavelength band to which radiated light belongs, wavelength band where reception sensitivity is high) may be provided, or a plurality of light emitters 110 and light receivers 120 having different optical characteristics may be provided.

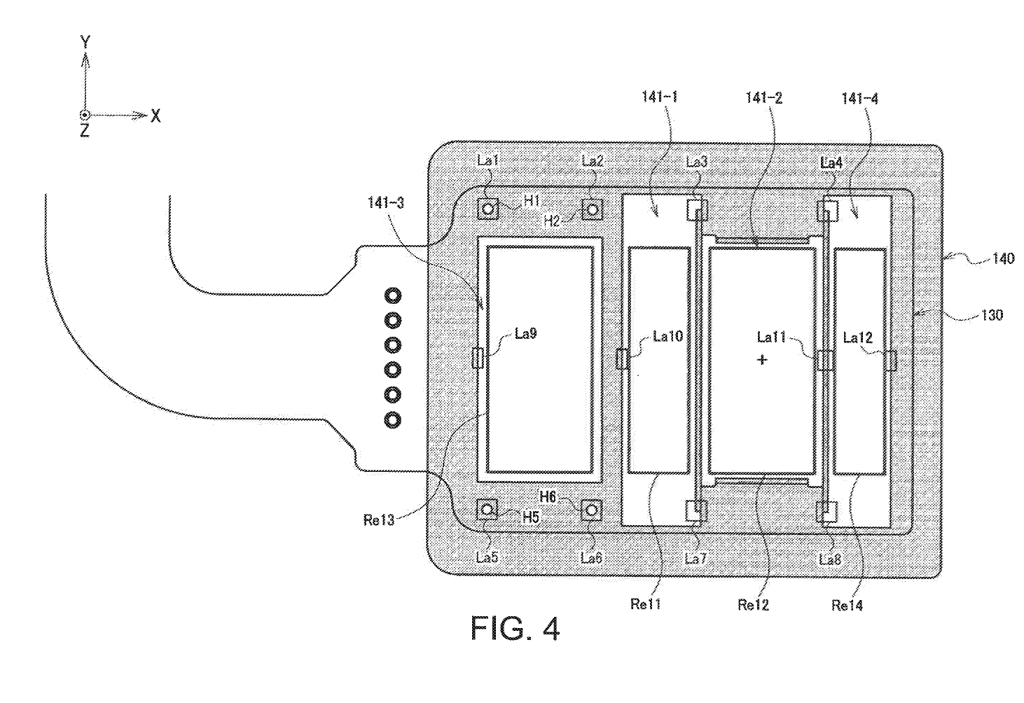

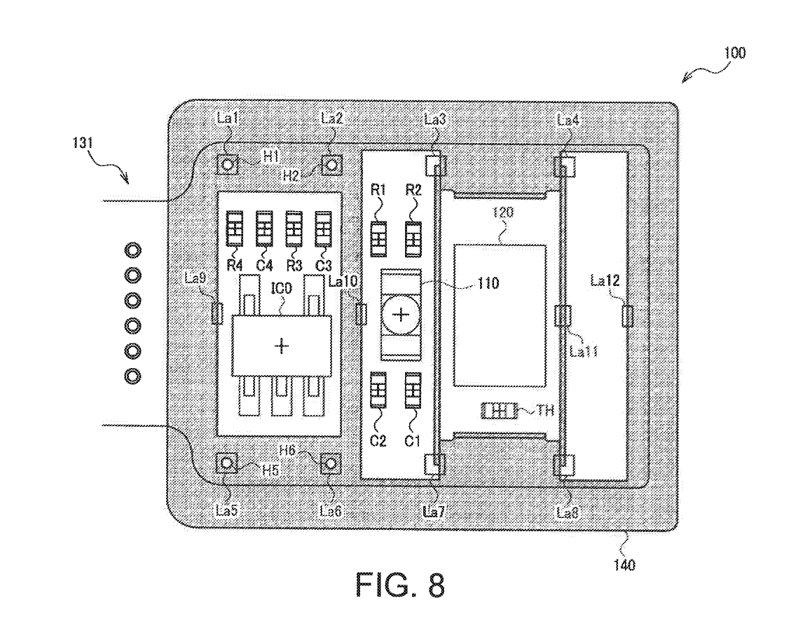

FIG. 2 is a side view of the optical sensor module 100 according to the present embodiment viewed in the direction along the mounting surface of the substrate 130. In FIG. 2, parts behind the other parts are also illustrated as required to clearly show the relationship among the heights of the parts. FIG. 2 also shows an integrated circuit IC0, which is accommodated in a detector 150, and a light blocker 160, and these parts will be described later in detail. In FIG. 2, it is assumed that a Z axis corresponds to the direction perpendicular to the mounting surface of the substrate 130 and X and Y axes correspond to directions along the mounting surface. Further, in FIG. 2, the lateral direction in the plane of view corresponds to the X axis, and the depth direction with respect to the plane of view corresponds to the Y axis. Instead, the X axis may correspond to the direction along one predetermined edge of the substrate 130, which has a roughly quadrangular shape, and the Y axis may correspond to the direction along one edge that intersects the predetermined edge, as will be described with reference to FIG. 4 and other figures. Still instead, in a case where the substrate 130 is provided with a connector section 131, which is connected to a main substrate (second substrate 70), the X axis may correspond to the direction from the connector section 131 toward a part mounting region Re1, and the Y axis may correspond to the direction perpendicular to the X axis, as will be described with reference to FIG. 8.

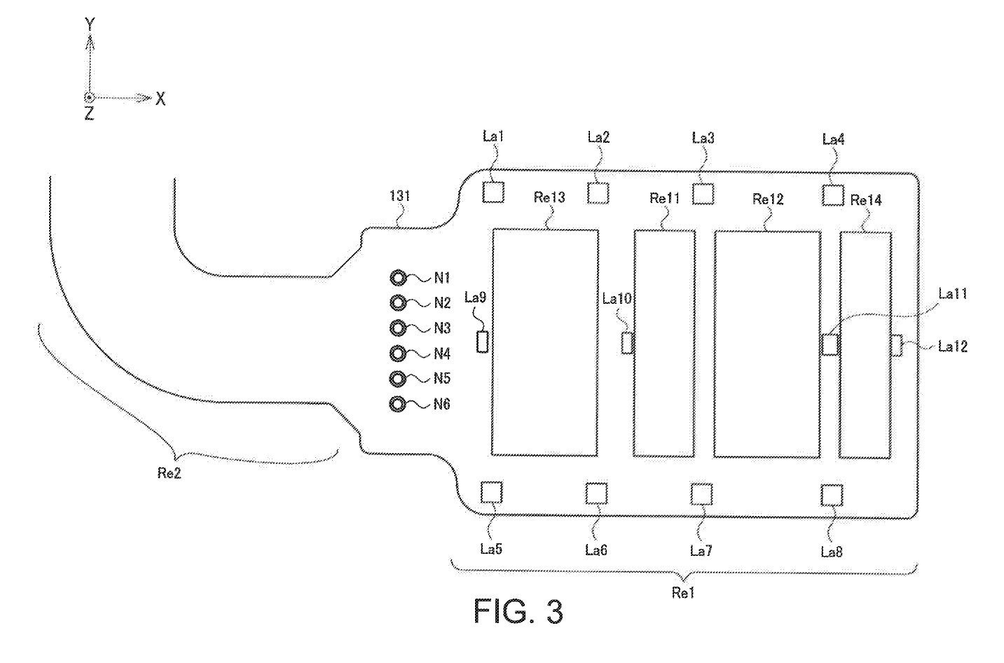

In the present embodiment, a deformable substrate is used as the substrate 130. The deformable substrate may be a flexible printed circuit (FPC). The flexible printed circuit is thinner than a solid substrate and is, for example, about 100 μm in thickness. That is, use of a deformable substrate allows reduction in thickness of the optical sensor module 100. Further, the flexible printed circuit can be used as wiring (flexible cable), as will be described later with reference, for example, to Re2 in FIG. 3. That is, when the optical sensor module 100 is connected to another substrate (for example, second substrate 70, which will be described later), it is unnecessary to provide a connection portion where the optical sensor module 100 is connected to a cable, and space saving is also achieved. A typical FPC is a printed board having a structure in which an electrically conductive foil is boned via an adhesive layer onto a base film that is a thin-film-shaped insulator, but the deformable substrate according to the present embodiment may be a substrate having a structure other than the structure described above.

Providing only the deformable substrate 130 results in insufficient strength, and the above-mentioned accuracy decrease due to deformation cannot therefore be suppressed. In this regard, in the present embodiment, the reinforcing plate 140, which reinforces the strength of the substrate 130, is provided. The reinforcing plate 140 is a member at least formed of a planar member extending in the direction along the mounting surface of the substrate 130 and connected (fixed, glued) to the mounting surface of the substrate 130 for suppression of deformation of the substrate 130. The reinforcement using the reinforcing plate 140 can thus suppress a decrease in detection accuracy resulting from deformation of the substrate 130.

It is not prohibited to connect the reinforcing plate 140 to the rear surface of the substrate 130, the surface opposite the mounting surface. In this case, the thickness h of the optical sensor module 100 is determined by the sum of the thickness h1 of the substrate 130, the thickness h3 of the reinforcing plate 140, and the thickness h2 of the parts mounted on the substrate 130. For example, in a case where a metal member is used as the reinforcing plate 140, even a sufficiently thin reinforcing plate 140 can provide strength to some degree, whereby the thickness of the optical sensor module 100 can be reduced even when the reinforcing plate 140 is fixed to the rear surface of the substrate 130.

The reinforcing plate 140 may instead be connected to the mounting surface of the substrate 130, as shown in FIG. 2. The reinforcing plate 140 is provided with a hole section 141, which does not interfere with the light emitter 110 or any other part when viewed in the Z-axis direction. That is, in the case where the reinforcing plate 140 is connected to the mounting surface of the substrate 130, the reinforcing plate 140 does not contribute to the entire thickness of the optical sensor module 100. As a result, the thickness h of the optical sensor module 100 is determined by the sum of the thickness h1 of the substrate 130 and the thickness h2 of the parts mounted on the substrate 130, whereby the thickness reduction effect provided by use of a thin deformable substrate as the substrate 130 can be enhanced. In first and second embodiments, which will be described later, the light blocker 160, which is part of the reinforcing plate 140, is the thickest portion of the optical sensor module 100 in some cases. However, since the light blocker 160 is characterized by blocking, for example, direct light to from the light emitter 110 to the light receiver 120, the height from the substrate surface (thickness) depends on the thickness of the light emitter 110 and other portions. That is, also in this case, the thickness of the entire optical sensor module 100 is still determined by h1+h2, as described above.

Specific examples of the configuration of the optical sensor module 100 according to the present embodiment will be described below, and examples of a specific apparatus including the optical sensor module 100 will then be described.

### 2. Examples of Configuration of Optical Sensor Module

As examples of the configuration of the optical sensor module 100, first to third embodiments will be described. In the optical sensor module 100 according to the first embodiment, the reinforcing plate 140 is a metal member, and part of the metal member forms the light blocker 160. In the optical sensor module 100 according to the second embodiment, the reinforcing plate 140 is a resin member, and part of the resin member forms the light blocker 160. In the optical sensor module 100 according to the third embodiment, the reinforcing plate 140 is a resin member, and the light blocker 160 formed of a metal member is formed as a member separate from the reinforcing plate 140.

2.1 First Embodiment (Case where Part of Reinforcing Plate Formed of Metal Member Forms Light Blocker)

FIG. 3 is a plan view of the substrate 130 accommodated in the optical sensor module 100 according to the first embodiment and viewed in the direction perpendicular to the mounting surface (viewed from the side where a target object is present when the optical sensor module is in operation). The substrate 130 is provided with a mounting region Re1 and a connector section 131 as well as a wiring region Re2, as shown in FIG. 3. The mounting region Re1 is a region where parts are mounted and includes a light emitter mounting region Re11, where the light emitter 110 is mounted, a light receiver mounting region Re12, where the light receiver 120 is mounted, an IC mounting region Re13, where an integrated circuit that forms the detector 150 is mounted, and an auxiliary mounting region Re14. The entire light emitter mounting region Re11 is not necessarily used to mount the light emitter 110, and the light emitter 110 may be mounted on part of the light emitter mounting region Re11, and other parts may be mounted on the remainder of the light emitter mounting region Re11. The same holds true for the light receiver mounting region Re12. The auxiliary mounting region Re14 may not be used to mount parts, and other parts that are not shown may be mounted on the auxiliary mounting region Re14 in FIG. 8 and other figures. Reference characters La1 to La12 shown in FIG. 3 represent solder lands used to be connect the substrate 130 to the reinforcing plate 140. The solder lands La1 to La12 will be described later in detail.

The substrate 130 is provided with the connector section 131, as shown in FIG. 3, which is electrically connected to the second substrate 70, which is provided with a processing section that performs processing based on a detection signal from the light receiver 120. The processing section in the description is, for example, a DSP (digital signal processor) or any other processor. In the case where the optical sensor module 100 is accommodated in the biological information detecting apparatus 200, the processing section carries out, for example, the process of computing biological information on the basis of the detection signal. In the case where the optical sensor module 100 is accommodated in a liquid consuming apparatus, the processing section carries out, for example, the process of determining the amount of remaining liquid on the basis of the detection signal.

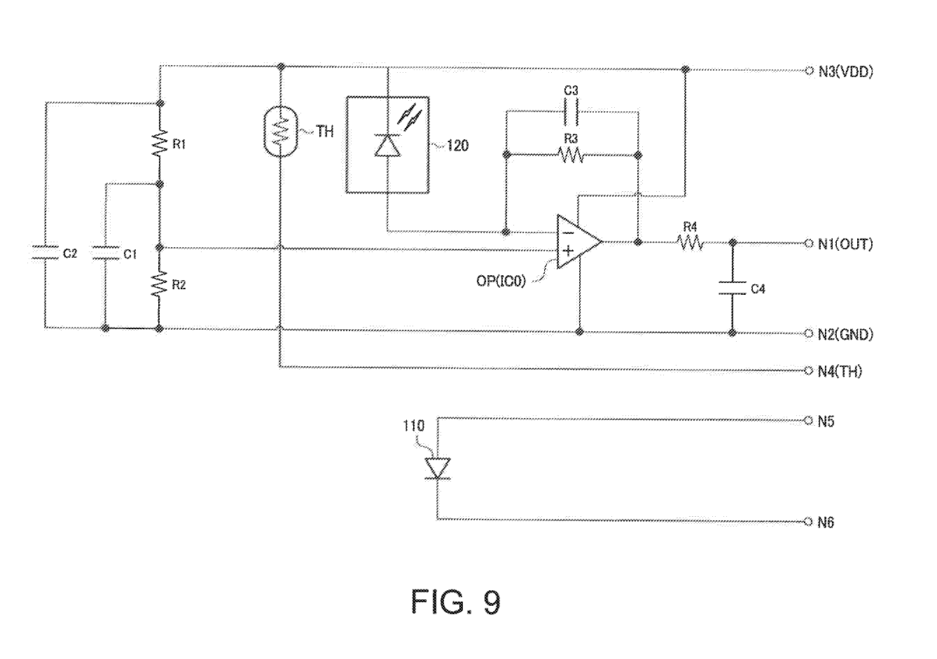

In the example shown in FIG. 3, the connector section 131 has a first terminal N1, via which the detection signal (OUT) is outputted to the processing section, a second terminal N2, to which low-potential-side reference potential (GND) is supplied, a third terminal N3, to which high-potential-side reference potential (VDD) is supplied, a fourth terminal N4, via which a temperature detection signal (TH) is outputted to the processing section, and fifth and sixth terminals N5, N6, via which a current signal is supplied to the light emitter 110. The terminals are electrically connected to the processing section via wiring lines provided in the wiring region Re2 of the substrate 130. The relationship of the terminals with the light emitter 110, the light receiver 120, the detector 150, and other components will be described later with reference to FIG. 9. The configuration of the connector section 131 is not limited to the configuration shown in FIG. 3 and can be changed in a variety of manners.

FIG. 4 is a plan view in a state in which the reinforcing plate 140 is fixed from the Z-axis positive direction side to the substrate 130 shown in FIG. 3. Part of the substrate 130 is covered with the reinforcing plate 140 and cannot therefore be visually recognized from the Z-axis positive direction side but is shown in FIG. 4 for convenience. In the example shown in FIG. 4, the reinforcing plate 140 is longer in both the X-axis and Y-axis directions than the mounting region Re1 (mounting surface) of the substrate 130 and is so provided as to cover the mounting region Re1 in a plan view viewed in the Z-axis positive direction. The reinforcing plate 140 can thus reinforce the substrate 130 over a sufficiently large area, whereby sufficient strength is provided. It is, however, noted that the reinforcing plate 140 and the mounting region Re1 of the substrate 130 may be so sized as to be equal to each other, as will be described later in the second embodiment. Further, the reinforcing plate 140 can even be smaller than the mounting region Re1 of the substrate 130 as long as sufficient strength can be provided.

The reinforcing plate 140 desirably does not overlap with the region where the light emitter 110 is mounted (light emitter mounting region Re11 in broad sense) or the region where the light receiver 120 is mounted (light receiver mounting region Re12 in broad sense) in a plan view viewed from the target object side. The reason for this is that the light emitter 110 irradiates primarily a target object with light and the light receiver 120 receives the light reflected off the target object. That is, since the Z-axis positive direction corresponds to the side where the target object is located when the optical sensor module 100 is in operation, light is undesirably blocked if the reinforcing plate 140 overlaps with the two regions described above when viewed in the Z-axis positive direction. Further, interference between parts and the reinforcing plate 140 when viewed in the Z-axis direction is not preferable from the viewpoint of thickness reduction, as described above with reference to FIG. 2.

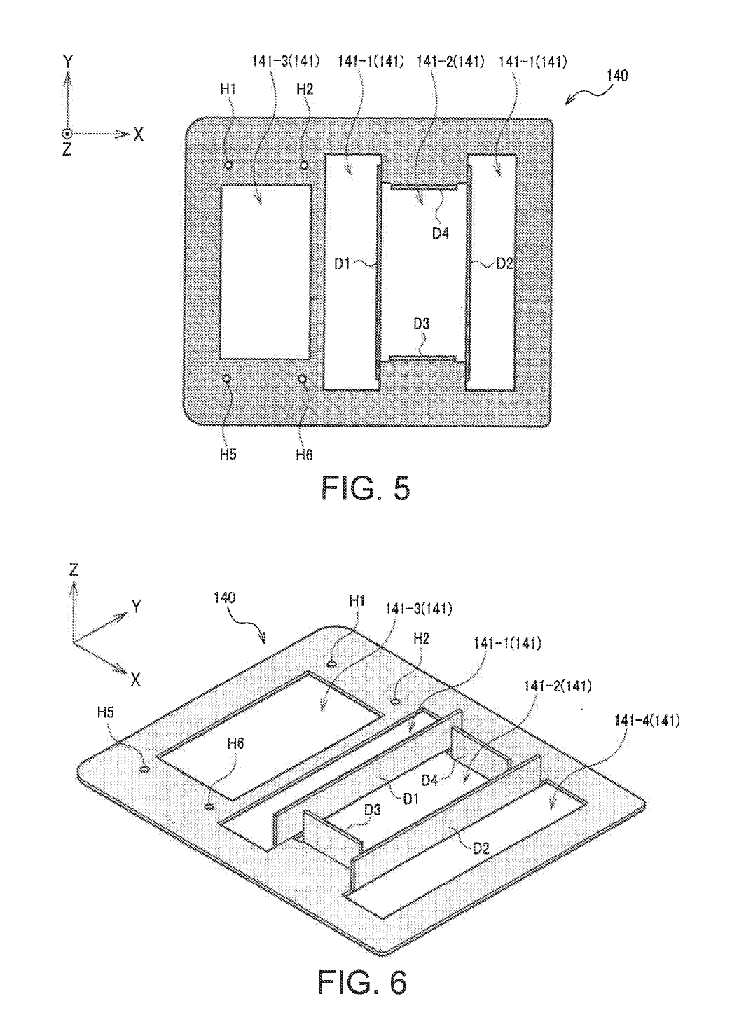

The reinforcing plate 140 therefore preferably has at least one hole section 141, which exposes the light emitter 110 and the light receiver 120 in the plan view viewed from the target object side. FIG. 5 is a plan view of the reinforcing plate 140 in the first embodiment. The reinforcing plate 140 may have, as the at least one hole section 141, a first hole section 141-1, which exposes the light emitter 110, and a second hole section 141-2, which exposes the light receiver 120, as shown in FIG. 5.

The state shown in FIG. 4 is achieved by connecting the reinforcing plate 140 shown in FIG. 5 to the substrate 130 from the Z-axis positive direction side. In FIG. 4, since the first hole section 141-1 contains the light emitter mounting region Re11, the first hole section 141-1 exposes the light emitter 110 irrespective of a specific mounting position of the light emitter 110 in the light emitter mounting region Re11. Similarly, since the second hole section 141-2 contains the light receiver mounting region Re12, the second hole section 141-2 exposes the light receiver 120.

In FIGS. 4 and 5, the reinforcing plate 140 has a third hole section 141-3, which contains the IC mounting region Re13, and a fourth hole section 141-4, which contains the auxiliary mounting region Re14. Therefore, also in the case where parts are mounted on the IC mounting region Re13 and the auxiliary mounting region Re14, interference between the parts and the reinforcing plate 140 is avoided.

In the case of the optical sensor module 100 including the light emitter 110 and the light receiver 120, the configuration including the light blocker 160 (light blocking wall) is widely known. In the optical sensor module 100, the light radiated from the light emitter 110, reflected off the target object, and received by the light receiver 120 is a target to be detected. Therefore, if direct light from the light emitter 110 is received by the light receiver 120, a signal resulting from the direct light forms noise. Since the light blocker 160 is a structure that blocks at least the direct light, providing the light blocker 160 allows an increase in the detection accuracy.

Even when the reinforcing plate 140 and the light blocker 160 are provided as separate members, no problem occurs from the viewpoint of thickness reduction, as will be described later in the third embodiment. In the present embodiment, however, part of the reinforcing plate 140 forms the light blocker 160, which blocks direct light from the light emitter 110 to the light receiver 120. The number of parts of the optical sensor module 100 can thus be reduced, whereby cost reduction and improvement in productivity are achieved. The light blocking used herein does not necessarily mean that the direct light is fully blocked but may mean that the intensity of the direct light is lowered to some degree.

FIG. 6 is a perspective view of the reinforcing plate 140 shown in FIG. 5. The reinforcing plate 140 has four surfaces D1 to D4, which surround the second hole section 141-2, as shown in FIG. 6. Reference characters D1 and D2 each represent a surface extending in the direction along a YZ plane, and reference characters D3 and D4 each represent a surface extending in the direction along an XZ plane. The light blocker 160 in the present embodiment is formed of D1 to D4.

The light blocker 160 is provided at least in a position between the first hole section 141-1 and the second hole section 141-2, as seen from D1 in FIG. 6. Since light that the light blocker 160 should primarily block is the direct light from the light emitter 110 to the light receiver 120, providing the light blocker 160 (D1) in a position between the two hole sections allows efficient light blockage. The reason for this is that since the light emitter 110 is mounted in a position corresponding to the first hole section 141-1 and the light receiver 120 is mounted in a position corresponding to the second hole section 141-2, the arrangement described above allows the light blocker 160 to be provided in a position between the light emitter 110 and the light receiver 120 in the plan view viewed from the target object side.

The light blocker 160 can also be provided in a position as well as the position between the two hole sections. For example, the light blocker 160 is provided in a position where the light blocker 160 surrounds the light receiver 120, as indicated by D2 to D4 in FIG. 6. This arrangement can prevent ambient light as well as the direct light from being incident on the light receiver 120.

In the present embodiment, the reinforcing plate 140 is formed of a metal member. A variety of approaches to formation of the light blocker 160 by using part of the reinforcing plate 140 are conceivable. For example, the light blocker 160 may be formed in sheet metal working.

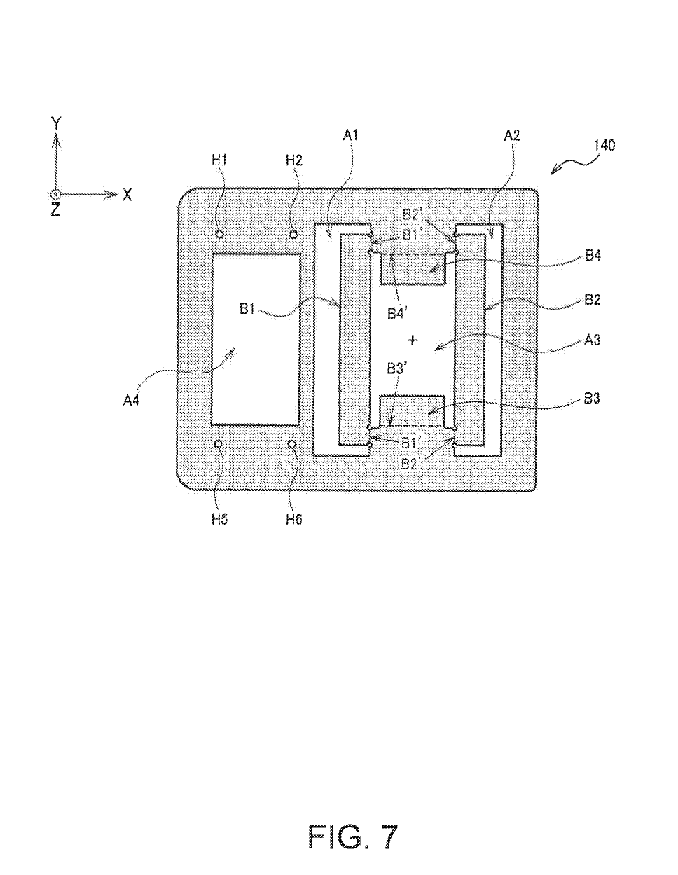

FIG. 7 is a plan view (development) of the reinforcing plate 140 before it is bent in sheet metal working (before light blocker 160 is formed). The reinforcing plate has a hole section A1, which has a rectangular shape having long edges in the Y-axis direction with a +Y-direction end portion and a −Y-direction end portion extending in the +X direction (roughly U-letter-like shape), and a hole section A2, which is provided on the +X-direction side of A1 and so shaped that A1 and A2 are symmetric with respect to the direction along the Y axis, as shown in FIG. 7. The reinforcing plate further has a hole section A3, which is located between A1 and A2 and has a rectangular shape having long edges in the Y-axis direction with a −X-direction end portion extending in the +Y and −Y directions and a +X-direction end portion extending in the +Y and −Y directions (roughly H-letter-like shape). The reinforcing plate further has a rectangular hole section A4 on the −X-direction side of A1 to A3.

In FIG. 7, B1 to B4 are portions that will form the light blocker 160. B1 is so bent by 90 degrees around the axis labeled with B1′ as to stand in the +Z direction. The bent B1 corresponds to D1 in FIG. 6. Similarly, B2 to B4 in FIG. 7 are so bent by 90 degrees around the axes labeled with B2′ to B4′ as to stand in the +Z direction. D2 to D4 in FIG. 6 can thus be formed.

The first hole section 141-1 is formed of a hole section A1 in FIG. 7, which has been open before the bending operation, and an open region produced by bending B1, as seen from comparison between FIGS. 5 and 7. Similarly, the second hole section 141-2 is formed of the hole section A3 in FIG. 7 and an open region produced by bending B3 and B4. The hole section 141 can thus be efficiently formed in sheet metal working for forming the light blocker 160.

Connection (fixation, gluing) between the substrate 130 and the reinforcing plate 140 will next be described. The reinforcing plate 140 is a configuration for suppressing deformation of the deformable substrate 130, such as a flexible printed circuit, and increasing the strength thereof, as described above. If the substrate 130 is configured to be deformable independently of the reinforcing plate 140, the meaning of provision of the reinforcing plate 140 deteriorates, and it is therefore necessary to appropriately connect the substrate 130 and the reinforcing plate 140 to each other.

The optical sensor module 100 therefore has connection portions where the substrate 130 and the reinforcing plate 140 to each other. In the present embodiment, it is assumed that the reinforcing plate 140 is a metal member. The connection portions may therefore connect the substrate 130 and the reinforcing plate 140 to each other with solder.

For example, the solder lands La1 to La12 are provided on the substrate 130, as shown in FIG. 3. Solder is applied onto the solder lands La1 to La12, and the reinforcing plate 140 is placed on the solder lands La1 to La12. In this state, a reflow furnace or any other apparatus is used to apply heat to the solder to melt it, whereby the substrate 130 and the reinforcing plate 140 are connected to each other. Since the substrate 130 and the reinforcing plate 140 can thus be connected to each other in the same approach to surface mounting, cost reduction and improvement in productivity are achieved. For example, part or entirety of the parts, such as the light emitter 110, can be mounted on the substrate 130 and the reinforcing plate 140 can be connected to the substrate at the same time. It is, however, noted that using an adhesive other than solder and other variations are conceivable.

The arrangement of the connection portions (the number of solder lands La1 to La12 and the positions thereof) can be set from a variety of viewpoints. For example, in consideration of the fact that the substrate 130 and the reinforcing plate 140 are fixed to each other with connection strength to some degree provided, the connection portions may be disposed in a peripheral portion of the substrate 130.

For example, the connection portions are disposed in a region along a first edge of the substrate 130 and a region along a second edge of the substrate 130 that faces the first edge. In the example shown in FIG. 3, the first and second edges represent the two edges extending in the direction along the X-axis. It is, however, noted that the region where the connection portions are disposed may instead be regions along the two edges in the Y-axis direction. The connection portion disposed in the region along the first edge corresponds to the solder lands La1 to La4, and the connection portion disposed in the region along the second edge corresponds to the solder lands La5 to La8. The arrangement described above allows the reinforcing plate 140 and the substrate 130 to be appropriately connected to each other, whereby the reinforcing plate 140 can be used to suppress deformation of the substrate 130. Further, La9 and La12 in FIG. 3 correspond to connection portions provided in the peripheral portion of the substrate 130 and therefore efficiently suppress deformation of the substrate, as La1 to La8 do.

The reason why deformation of the substrate 130 poses a problem is that swing motion of the light emitter 110 and the light receiver 120 causes noise to occur, as described above. From this point of view, high priority on suppression of deformation is given to the region where the light emitter 110 and the light receiver 120 are provided, and relatively low priority on suppression of deformation is given to the other regions, such as the IC mounting region Re13.

Therefore, in the plan view viewed from the target object side (plan view viewed in direction perpendicular to mounting surface of the substrate 130, plan view viewed in Z-axis positive direction), the plurality of connection portions are preferably so provided as to surround the light emitter 110 and the light receiver 120. In the example shown in FIG. 3, the connection portions so provided as to surround the light emitter 110 and the light receiver 120 correspond to the solder lands La2 to La4, La6 to La8, La10, and La11.

The arrangement described above allows the substrate 130 and the reinforcing plate 140 to be fixed to each other in positions that surround the light emitter 110 and the light receiver 120, whereby swing motion of the light emitter 110 and the light receiver 120 can be efficiently suppressed.

Further, from the viewpoint of suppression of swing motion of the light emitter 110 and the light receiver 120, in the plan view viewed from the target object side, the reinforcing plate 140 may be so provided as to contain the light emitter 110 and the light receiver 120. In the example shown in FIG. 4, the light emitter 110 and the light receiver 120 are provided in the first hole section 141-1 and the second hole section 141-2 of the reinforcing plate 140, respectively, and the member that forms the reinforcing plate 140 is present along the entire circumference of each of the hole sections (all directions over 360 degrees). It is, however, noted that the containment in the present embodiment is not limited to the state in which the entire circumference is surrounded and may be a state in which the member that forms the reinforcing plate 140 is not present in part of the directions. Since swing motion of the regions that form the substrate 130 and surround the light emitter 110 and the light receiver 120 is thus suppressed, swing motion of the light emitter 110 and the light receiver 120 can also be efficiently suppressed.

To enhance the thickness reduction effect, portions corresponding to the connection portions may be processed. The connection between the substrate 130 and the reinforcing plate 140 is achieved, for example, by using solder, as described above. In this case, in the plan view viewed in the direction perpendicular to the substrate 130, assuming that the reinforcing plate 140 is so connected to the substrate 130 as to cover the entire solder lands, the solder applied onto the solder lands are left in the region between the substrate 130 and the reinforcing plate 140 after the connection. In a case where excessive solder is applied onto the solder lands, the solder left in the region between the substrate 130 and the reinforcing plate 140 has a thickness, and the thickness of the optical sensor module 100 is therefore undesirably likely to increase.

In contrast, in the case where at least part of the solder lands overlaps with the hole section 141 of the reinforcing plate 140 in the plan view, such as La3, La4, and La7 to La12 in FIG. 4, the excessive solder moves (escapes) in the direction toward the hole section. That is, the increase in the thickness due to the solder can be suppressed as long as a space that allows the solder to escape is present.

A structure that allows the solder to escape may therefore be provided in the position corresponding to each of the solder lands. In the example shown in FIGS. 5 to 7, hole sections H1, H2, H5, and H6 that allow the solder to escape are provided in the positions corresponding to the solder lands La1, La1, La5, and La6. In the state after the connection is established, since a solder land Lai overlap with a hole section Hi (i=1, 2, 5, and 6) in the plan view, as shown in FIG. 4, excessive solder applied onto the solder lands is allowed to escape through the hole sections in the direction toward the +Z side of the reinforcing plate 140. A larger area of each of the hole sections H1, H2, H5, and H6 allows the solder to more readily escape, but the connection area decreases and the connection strength decreases accordingly. Specific shape and size of the hole sections may be determined in consideration of a variety of conditions. Further, since the reinforcing plate 140 only needs to be provided with a structure that allows the solder to escape (solder escape portion), the structure is not limited to a hole. For example, as the solder escape portion, a cutout or any other structure may be used.

FIG. 8 is a plan view of the optical sensor module 100 with the light emitter 110, the light receiver 120, and other parts mounted on the substrate 130. A side view of the optical sensor module 100 in FIG. 8 viewed from the Y-axis negative direction corresponds to FIG. 2. In the example shown in FIG. 8, the light emitter 110, the light receiver 120, resistors R1 to R4, capacitors C1 to C4, the integrated circuit IC0, which corresponds to an operational amplifier OP, and a temperature sensor TH are mounted on the substrate 130. The light emitter 110, the resistors R1 and R2, and the capacitors C1 and C2 are mounted on the light emitter mounting region Re11. The light receiver 120 and the temperature sensor TH are mounted on the light receiver mounting region Re12. The resistors R3 and R4, the capacitors C3 and C4, and the integrated circuit IC0 are mounted in the IC mounting region Re13. It is, however, noted that the arrangement of the parts can be changed in a variety of manners. Further, some of the parts shown in FIG. 8 can be omitted, and another part can be added to the parts shown in FIG. 8.

FIG. 9 shows an example of the circuit diagram of the optical sensor module 100. The anode of the light emitter 110 (LED) is connected to the fifth terminal N5, and the cathode of the light emitter 110 is connected to the sixth terminal N6. The current signal is thus provided to the light emitter 110 via the connector section 131.

The temperature sensor TH is provided in a position between the third terminal N3, to which the high-potential-side reference potential VDD is supplied, and the fourth terminal N4 and outputs the temperature detection signal via the fourth terminal N4.

The cathode of the light receiver 120 (photodiode, PD) is connected to the third terminal N3, and the anode of the light receiver 120 is connected to the inverted input terminal of the operational amplifier OP. The current signal produced when the light receiver 120 receives light is inputted to the inverted input terminal of the operational amplifier OP.

The resistors R1 and R2 are provided in series between the third terminal N3 and the second terminal N2. The resistors R1 and R2 divide the voltage corresponding to the potential difference between VDD and GND to produce reference voltage, and the produced reference voltage is inputted to the non-inverted input terminal of the operational amplifier OP. Specifically, the node between the resistors R1 and R2 is connected to the non-inverted input terminal of the operational amplifier OP. The capacitor C1 is connected in parallel to the resistor R2, and the capacitor C2 is connected in parallel to the resistors R1 and R2. The capacitors C1 and C2 are each a capacitor for stabilization.

The two power source terminals of the operational amplifier OP are connected to the second terminal N2 and the third terminal N3, respectively, and the operational amplifier OP operates by using signals through the power source terminals as a power source. The resistor R3 and the capacitor C3 are provided in parallel to each other between the output terminal and the non-inverted input terminal of the operational amplifier OP. The operational amplifier OP, the resistor R3, and the capacitor C3 form a transformer impedance amplifier (TIA), which is an amplifier that converts current into voltage. That is, the operational amplifier OP outputs a signal representing the current having been outputted from the light receiver 120 and having undergone the voltage conversion and amplification.

The resistor R4 is provided in a position between the output terminal of the operational amplifier OP and the first terminal N1, and the capacitor C4 is provided in a position between a node of the resistor R4, the node on the side facing the first terminal N1, and the second terminal N2. The resistor R4 and the capacitor C4 form a lowpass filter, and a signal representing the signal having been outputted from the operational amplifier OP and having undergone lowpass filtering is outputted as an output signal OUT via the first terminal N1.

The optical sensor module 100 includes the detector 150, which includes at least an amplification section that amplifies the detection signal from the light receiver 120, as shown in FIGS. 8 and 9, and the detector 150 may be provided on the substrate 130.

The amplification section used herein is achieved by the transformer impedance amplifier described above. In the example shown in FIGS. 8 and 9, the detector 150 also includes the lowpass filter. The optical sensor module 100 can thus output a signal having been outputted from the light receiver 120 and having undergone the amplification and other types of processing. The configuration of the detector 150 can be changed in a variety of manners. For example, in a case where the processing section of the second substrate 70 (main substrate) includes a lowpass filter, which is an antialiasing filter, and an A/D conversion circuit, the lowpass filter in the optical sensor module 100 may be omitted. Instead, a configuration other than the amplifier and the lowpass filter may be added to the detector 150.

In the present embodiment, L1<L2 and L1<L3 are satisfied, where L1 is the distance from the connector section 131 to the detector (transformer impedance amplifier, lowpass filter), L2 is the distance from the connector section 131 to the light emitter 110, and L3 is the distance from the connector section 131 to the light receiver 120, as shown in FIG. 8. The output signal from the optical sensor module 100 is outputted from the detector 150. Therefore, satisfying L1<L2 and L1<L3 allows the distance between the connector section 131 and the detector 150 to be shortened, whereby wiring in the substrate 130 can be readily performed. In the example shown in FIG. 8, L1<L2<L3 is satisfied, but not necessarily. Instead, a variation in which L2<L1 and L3<L1 are satisfied is conceivable, as will be described later in the second and third embodiments. The distances L1 to L3 may each be a distance with respect to the center of each involved part in the plan view viewed from the target object side. For example, in FIG. 8 and other figures, since the first to sixth terminals N1 to N6 of the connector section 131 are arranged on a straight line in the direction along the Y axis, the distances described above are each a distance in the X-axis direction from the straight line to the center of the corresponding part. The distance L2 is the distance from the straight line described above to the center of the light emitter 110, and the distance L3 is the distance from the straight line described above to the center of the light receiver 120. Since the detector 150 includes the integrated circuit IC0, the resistors R3 and R4, the capacitors C3 and C4, and other components, the center of any of the parts may, for example, be used as a representative point, or the center of the IC mounting region Re13 may be used. The distance reference is not limited to the center of a part and can be changed in a variety of manners, such as a predetermined end point and another reference point.

As described above as the first embodiment, the reinforcing plate 140 may be a metal member. The metal member used herein is, for example, nickel silver, which is an alloy of copper, zinc, and nickel. It is, however, noted that the metal member may instead be brass, which is an alloy of copper and zinc, stainless steel, which is alloy steel containing iron and chromium, or any other metal member.

Using a metal member allows a member thinner than the resin member in the second embodiment to have necessary strength and other advantageous properties. Further, connecting the metal member to the ground allows the metal member to provide a shielding effect. For example, in the case where the low-potential-side reference potential is the ground, connecting the reinforcing plate 140, which is a metal member, to the second terminal N2 allows the reinforcing plate 140 to be used as a shielding member.

2.2 Second Embodiment (Case where Part of Reinforcing Plate Formed of Resin Member Forms Light Blocker)

A second embodiment will next be described. The reinforcing plate 140 in the present embodiment is a resin member. A resin member can be formed in injection molding using a die. Therefore, also in the case where part of the reinforcing plate 140 forms the light blocker 160, a shape that satisfies requirements can be readily produced in bulk.

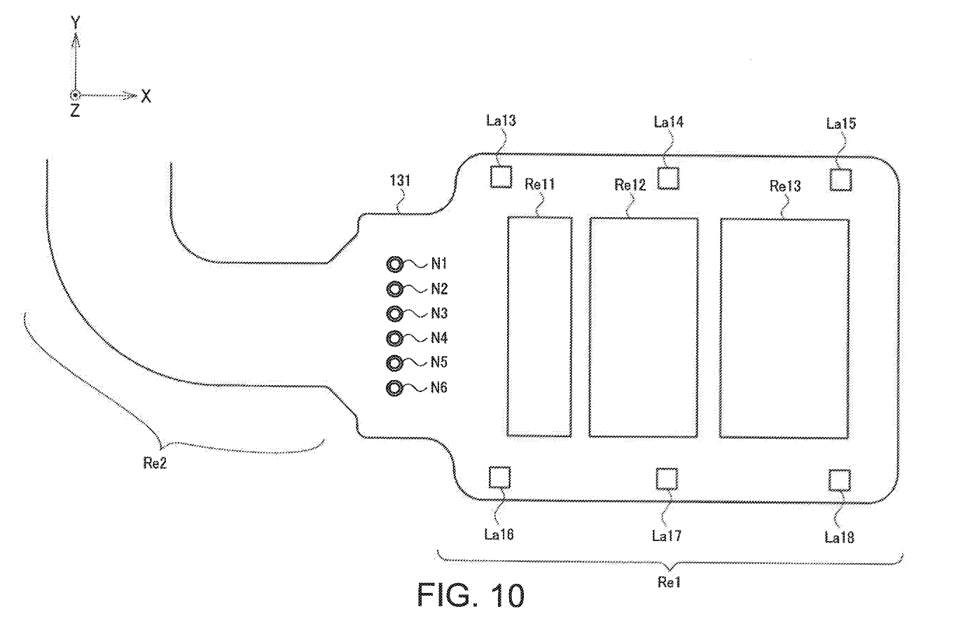

FIG. 10 is a plan view of the substrate 130 accommodated in the optical sensor module 100 according to the second embodiment and viewed in the direction perpendicular to the mounting surface. The substrate 130 is provided with the mounting region Re1 and the connector section 131 as well as the wiring region Re2, as in the first embodiment. The mounting region Re1 includes the light emitter mounting region Re11, the light receiver mounting region Re12, and the IC mounting region Re13. In the second embodiment, the light emitter mounting region Re11, the light receiver mounting region Re12, and the IC mounting region Re13 are arranged along the +X direction in the ascending order of the distance to the connector section 131. Reference characters La13 to La18 represent solder lands corresponding to connection portions, as in the first embodiment.

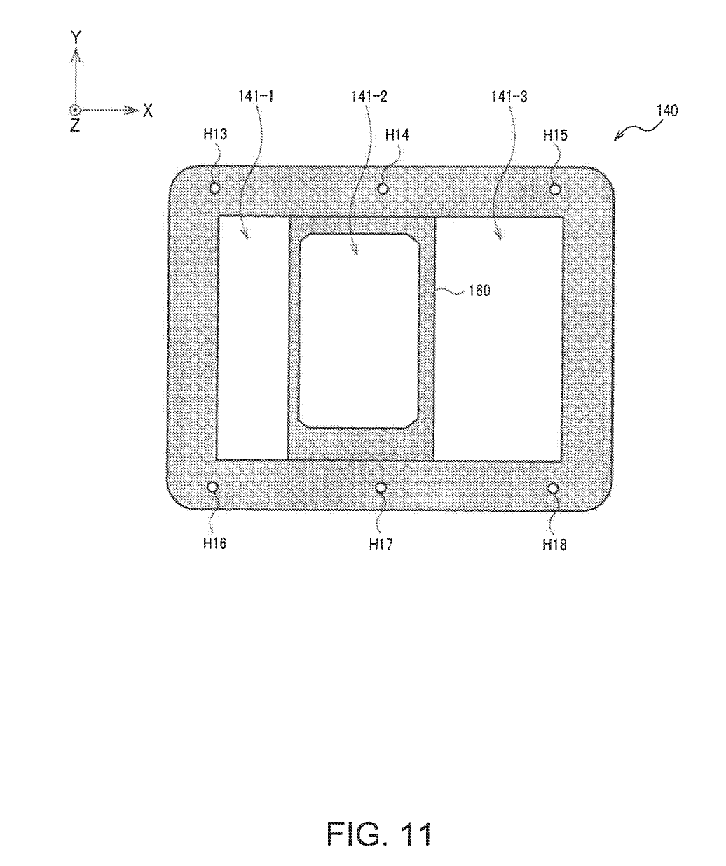

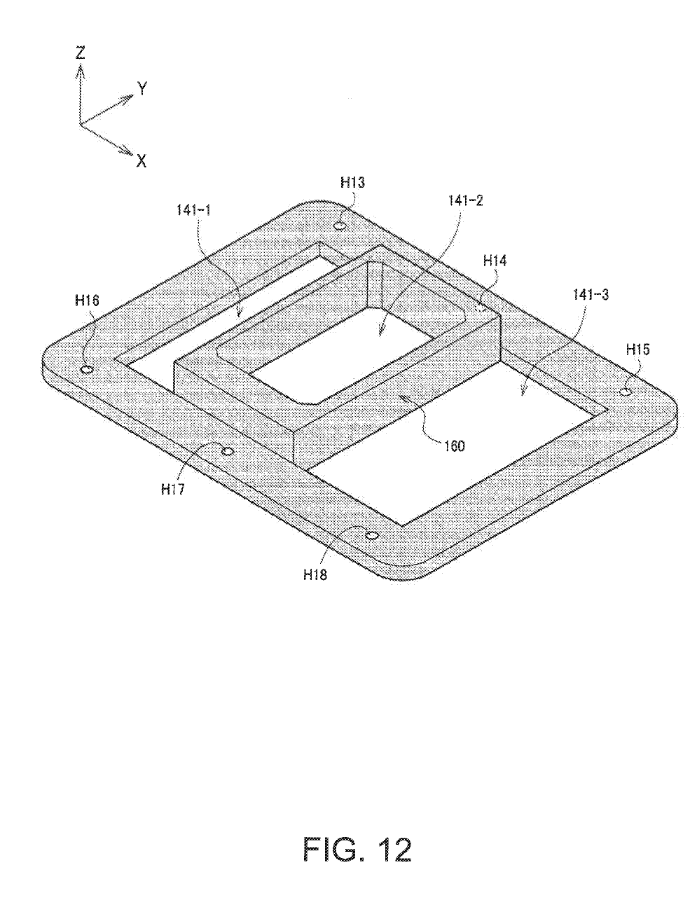

FIGS. 11 and 12 describe the shape of the reinforcing plate 140 in the present embodiment, which is a resin member. FIG. 11 is a plan view, and FIG. 12 is a perspective view.

The reinforcing plate 140, which is a resin member, has a first hole section 141-1, a second hole section 141-2, and a third hole section 141-3, as shown in FIGS. 11 and 12. The three hole sections are arranged in the +X direction in the order of the first hole section 141-1, the second hole section 141-2, and the third hole section 141-3 in correspondence with Re11 to Re13 in FIG. 10.

The region around the second hole section 141-2 in the plane view viewed from the Z-axis positive direction side is higher in the Z-axis direction than the other portions of the reinforcing plate 140, as shown in FIG. 12. The resin member of the portion corresponding to the region around the second hole section 141-2 forms a wall-shaped light blocker 160, which surrounds the second hole section 141-2, as seen from FIG. 12.

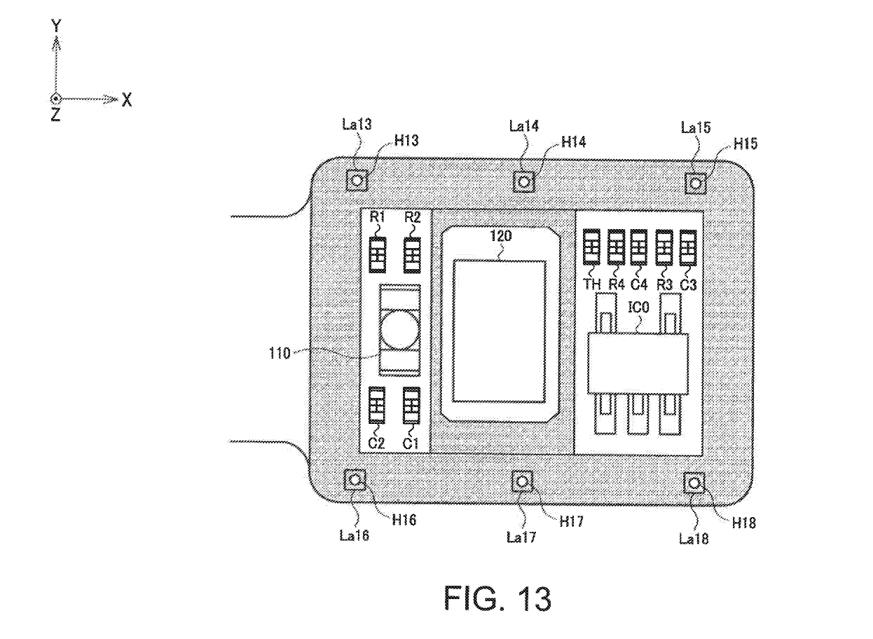

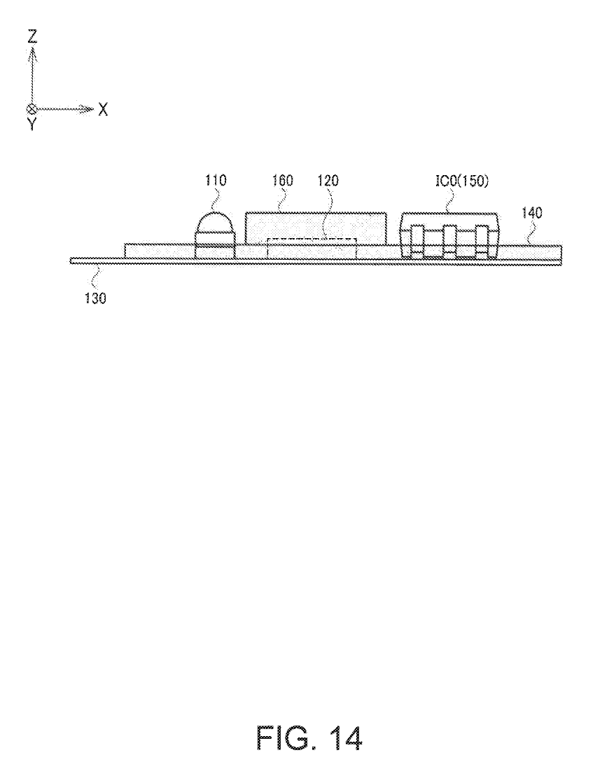

FIG. 13 is a plan view of the optical sensor module 100 in which the reinforcing plate 140 shown in FIGS. 11 and 12 and other parts are mounted on the substrate 130 and which is viewed from the target object side. FIG. 14 is a side view of the optical sensor module 100 in FIG. 13 viewed in the −Y direction.

Since each part, including the light emitter 110 and the light receiver 120, is disposed in any of the first to third hole sections 141-1 to 141-3 of the reinforcing plate 140, as shown in FIGS. 13 and 14, the reinforcing plate 140 does not interfere with the parts. Further, since the light blocker 160 is provided around the light receiver 120, incidence of light that causes noise to occur on the light receiver 120 can be suppressed.

Among recent resin members, a resin member having a metal terminal provided in part thereof is widely known. Using the metal terminal portion as the connection portion allows the connection between the substrate 130 and the reinforcing plate 140 to be achieved, for example, by using solder, as in the first embodiment. It is, however noted that the connection may be achieved by using an adhesive or any other material other than solder. Further, FIG. 13 and other figures show the case where six connection portions are provided, but the arrangement of the connection portions can be set from a variety of viewpoints, as in the first embodiment. Moreover, hole sections H13 to H18, each of which is a structure that allows solder to escape, may be provided in correspondence with the solder lands La13 to La18, as shown in FIGS. 11 and 12, also as in the first embodiment.

### 2.3 Third Embodiment (Combination of Reinforcing Plate Formed of Resin Member and Light Blocker Formed of Metal Member)

The first and second embodiments have been described with reference to the case where part of the reinforcing plate 140 forms the light blocker 160, but not necessarily. The optical sensor module 100 may include a light blocker 160 that is formed as a member separate from the reinforcing plate 140 and blocks direct light from the light emitter 110 to the light receiver 120. For example, the optical sensor module 100 includes a light blocker 160 formed of a metal member and a reinforcing plate 140 formed of a resin member. The third embodiment will be described below in detail. The substrate 130 will be described with reference to the same configuration as that in FIG. 10.

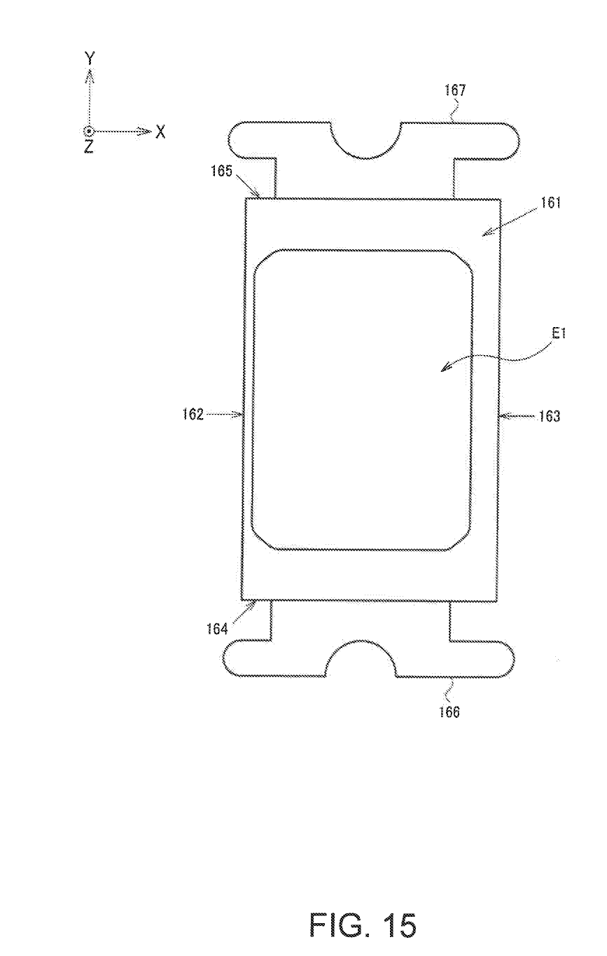

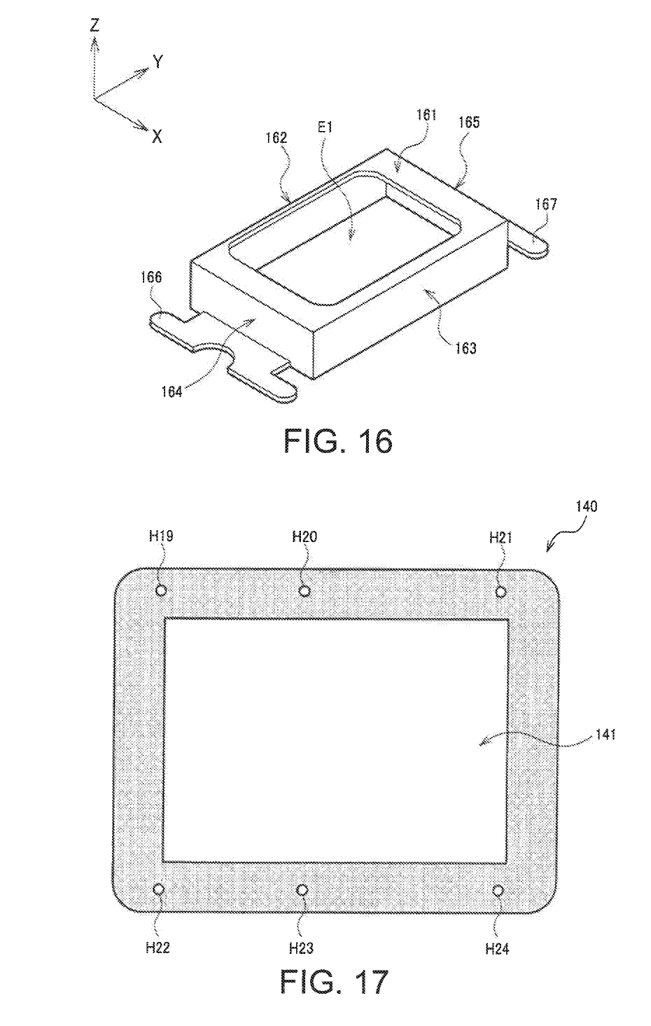

FIGS. 15 and 16 show an example of the structure of the light blocker 160 formed of a metal member. FIG. 15 is a plan view of the light blocker 160 viewed in the direction perpendicular to the mounting surface of the substrate 130 on which the light blocker 160 is to be mounted. FIG. 16 is a perspective view of the light blocker 160.

The light blocker 160 has a first metal surface 161, which is a surface extending in the direction along an XY plane (mounting surface of substrate 130 on which light blocker 160 is to be mounted) and having an opening E1, as shown in FIGS. 15 and 16. The light blocker 160 further has a second metal surface 162, a third metal surface 163, a fourth metal surface 164, and a fifth metal surface 165, which are disposed in a direction that intersects the first metal surface 161 and form the side surfaces of the light blocker 160. The second metal surface 162 and the third metal surface 163 are each a surface extending in the direction along a YZ plane, and the fourth metal surface 164 and the fifth metal surface 165 are each a surface extending in the direction along an XZ plane.

The light blocker 160 further has a sixth metal surface 166, which is a surface extending in the direction along an XY plane and connected to the fourth metal surface 164, and a seventh metal surface 167, which is a surface extending in the direction along the XY plane and connected to the fifth metal surface 165.

FIG. 17 is a plan view of the reinforcing plate 140 formed of a resin member. The reinforcing plate 140 has one hole section 141, as shown in FIG. 17. That is, one hole section 141 that contains both the light emitter mounting region Re11 and the light receiver mounting region Re12 is provided. It is, however, noted that a variation in which a hole section that exposes the light emitter 110 and a hole section that exposes the light receiver 120 are separately provided can be employed also in the present embodiment.

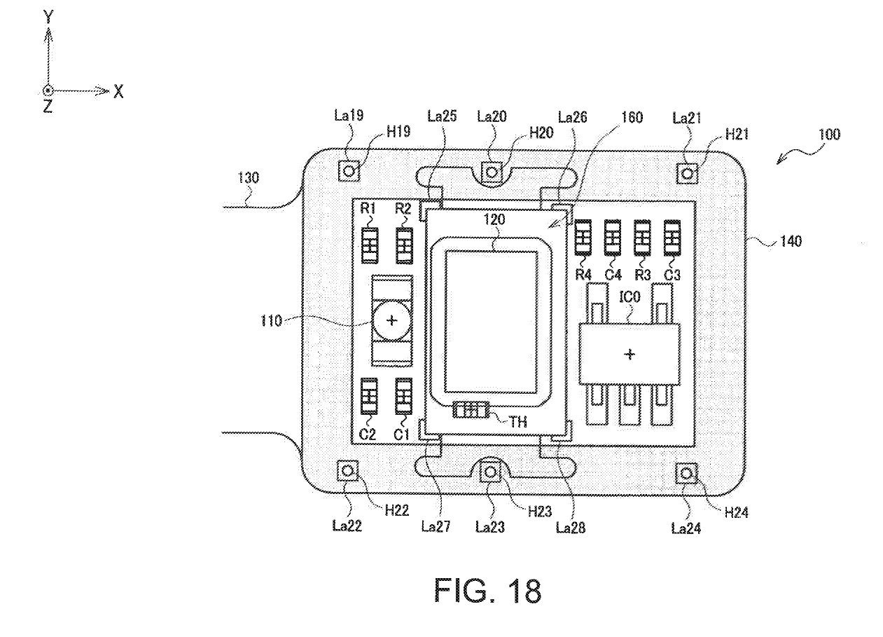

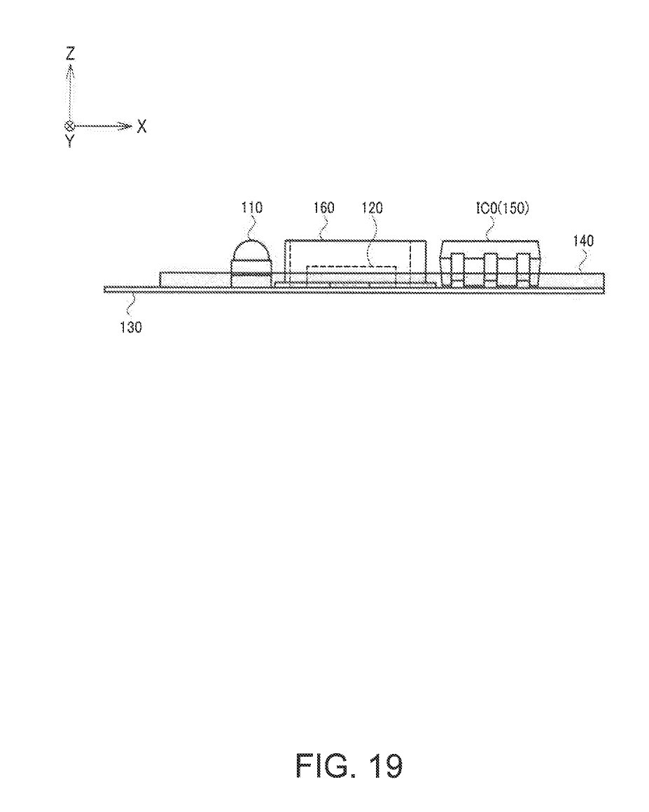

FIG. 18 is a plan view of the optical sensor module 100 in which the reinforcing plate 140, the light blocker 160, and other parts are mounted on the substrate 130 and which is viewed from the target object side. FIG. 19 is a side view of the optical sensor module 100 in FIG. 18 viewed in the −Y direction. Providing the hole section 141 prevents the light emitter 110 and other parts from interfering with the reinforcing plate 140, as shown in FIGS. 18 and 19. Further, since the light receiver 120 is located in the position corresponding to the opening E1 of the first metal surface 161 of the light blocker 160, incidence of light other than the light passing through the opening E1 of the first metal surface 161 on the light receiver 120 can be suppressed.

In the present embodiment, the light blocker 160 is placed on the substrate 130 from the +Z side, and the reinforcing plate 140 is further placed on the substrate 130 from the +Z side. The sixth metal surface 166 and the seventh metal surface 167 of the light blocker 160 are thus sandwiched between the substrate 130 and the reinforcing plate 140, whereby the light blocker 160 can be appropriately fixed.

Solder lands La19 to La24 for connecting the reinforcing plate 140 to the substrate 130 and solder lands La25 to La28 for connecting the light blocker 160 to the substrate are shown, but the number of solder lands and the arrangement thereof can be changed in a variety of manners, as in the first and second embodiments. Further, H19 to H24 corresponding to La19 to La24 are shown as the hole sections, each of which is a solder escape portion, and the solder escape portions can also be changed in a variety of manners.

### 3. Examples of Apparatus Including Optical Sensor Module

The approach in the present embodiment can be applied to the biological information detecting apparatus 200 including the optical sensor module 100 described above.

FIG. 20 is an exploded view of the biological information detecting apparatus 200 including the optical sensor module 100. The biological information detecting apparatus 200 includes a first enclosure 31 and a second enclosure 32, and the first enclosure 31 and the second enclosure 32 form an enclosure 30 (main body), as shown in FIG. 20. In the enclosure 30 are provided the optical sensor module 100, the battery 60, the second substrate 70 (main substrate), and the OLED (organic light emitting diode) panel 80.

The battery 60 supplies electric power that allows each portion of the biological information detecting apparatus 200 to operate. The second substrate 70 is provided with the processing section and other components, and the processing section carries out the process of detecting biological information and other processes on the basis of a signal from the optical sensor module 100. The processing section may further perform battery control and notification control using the OLED panel 80 and other components. The OLED panel 80 is a light emitter for notification to the user. For example, part of the first enclosure 31 is formed of a light transmissive member, and light emitted from the OLED panel 80 is visually recognized from the outside.

The biological information detecting apparatus 200 shown in FIG. 20 may, for example, be a wearable (wristwatch-shaped) instrument worn around the user's arm. In this case, a band section for fixing the enclosure 30 to the user's arm is connected to end portions of the enclosure 30.

FIGS. 21 and 22 show an example of the exterior appearance of a biological information detecting apparatus 200 different from the example in FIG. 20. The biological information detecting apparatus 200 includes the enclosure 30 and a band section 10 for fixing the enclosure 30 to the user's body (wrist in narrow sense), and the band section 10 is provided with fitting holes 12 and a buckle 14, as shown in FIG. 21. The buckle 14 is formed of a buckle frame 15 and a locking section (projection rod) 16.

FIG. 21 is a perspective view of the biological information detecting apparatus 200 which is in a state in which the fitting hole 12 and the locking section 16 are used to fix the band section 10 and which is viewed from the side facing the band section 10 (side facing subject-side surface of enclosure 30 worn on user). The biological information detecting apparatus 200 shown in FIG. 21 is worn on the user by inserting the locking section 16 of the buckle 14 into any of the plurality of fitting holes 12 provided in the band section 10. The plurality of fitting holes 12 are provided along the longitudinal direction of the band section 10, as shown in FIG. 21.

The optical sensor module 100 is provided in the subject-side surface of the enclosure 30 of the biological information detecting apparatus 200 worn on the user.

FIG. 22 shows the biological information detecting apparatus 200 worn on the user and which is viewed from the side where a display section 50 is provided. The biological information detecting apparatus 200 according to the present embodiment includes the display section 50 in the position corresponding to the dial of a typical wristwatch or in a position where a numeral or an icon can be visually recognized, as seen from FIG. 22. In the state in which the biological information detecting apparatus 200 is worn on the user, the surface that forms the enclosure 30 and is shown in FIG. 21 is in intimate contact with the subject, and the display section 50 is located in a position readily visually recognized by the user.

The approach in the present embodiment is applicable to an electronic instrument 300 including the optical sensor module 100 described above. The electronic instrument 300 can be achieved by a variety of apparatus and is conceivably, for example, a printing apparatus and a distance measuring apparatus.

FIG. 23 is a perspective view showing key parts of a printing apparatus (liquid consuming apparatus) including the optical sensor module 100. The X, Y, and Z axes in FIG. 23 are perpendicular to one another, and in a typical use posture of the printing apparatus, it is assumed that the forward direction of the printing apparatus is the X direction and the vertical direction is the Z direction. The coordinate system in FIG. 23 is a coordinate system set in the printing apparatus and may not coincide with the coordinate system of the optical sensor module 100 described above with reference to FIG. 2 and other figures.

The printing apparatus includes ink cartridges IC1 to IC4 (liquid containers, liquid accommodating containers), a carriage 320 including a holder 321, which detachably accommodates the ink cartridges IC1 to IC4, a cable 330, a sheet feeding motor 340, a carriage motor 350, a carriage driving belt 355, and the optical sensor module 100.

The ink cartridges IC1 to IC4 each accommodates single-color ink (liquid, printing material). The ink cartridges IC1 to IC4 are detachably loaded in the holder 321. A head is provided on the −Z-side surface of the carriage 320. The ink supplied from each of the ink cartridges IC1 to IC4 is discharged from the head toward a recording medium. The recording medium is, for example, a printing sheet. The carriage motor 350 drives the carriage driving belt 355 to move the carriage 320 in the ±Y directions.

The optical sensor module 100 outputs a signal for detecting the state of remaining ink in each of the ink cartridges IC1 to IC4. Specifically, the light emitter 110 radiates light to a prism provided in each of the ink cartridges IC1 to IC4, and the light receiver 120 receives the light reflected off the prism and converts the light into an electric signal.

For example, let θ1 be the critical angle at which total reflection occurs and θ2 be the angle of incidence of the light on the prism, and the printing apparatus is so set that θ1>θ2 is satisfied in a case where ink is left in an ink cartridge, whereas θ2>θ1 is satisfied in a case where no ink is left. The critical angle θ1 is determined in accordance with the material of the prism and the characteristics of the ink.

In the setting described above, since no total reflection occurs when ink is left, the majority of the light enters the ink cartridge, and the signal received by the light receiver 120 therefore decreases. On the other hand, since total reflection occurs in the prism when no ink is left, the signal received by the light receiver 120 relatively increases. Detecting the difference between the signal levels allows detection of the amount of remaining ink using the optical sensor module 100.

The embodiments and variations to which the invention is applied have been described, but the invention is no limited directly to the embodiments or variations and can be embodied in the implementation of the invention with the components in the embodiments and variations changed to the extent that the changes de not depart from the substance of the invention. Further, the plurality of components disclosed in the embodiments and variations described above can be combined with one another as appropriate to achieve a variety of forms of invention. For example, some of the components described in the embodiments and variations may be omitted. Further, the components described in the different embodiments and variations may be combined with one another as appropriate. A term described at least once in the specification or the drawings along with a different term having a boarder meaning or the same meaning can be replaced with the different term anywhere in the specification or the drawings. A variety of variations and applications are thus conceivable to the extent that they do not depart from the substance of the invention.

What is claimed is:

1. An optical sensor module comprising:
a light emitter that radiates light to a target object;
a light receiver that receives light from the target object;
a deformable substrate on which the light emitter and the light receiver are provided; and
a reinforcing plate that reinforces strength of the substrate.

2. The optical sensor module according to claim 1,
wherein part of the reinforcing plate forms a light blocker that blocks direct light from the light emitter to the light receiver.

3. The optical sensor module according to claim 1,
further comprising a light blocker that is formed as a member separate from the reinforcing plate and blocks direct light from the light emitter to the light receiver.

4. The optical sensor module according to claim 1,
further comprising a connection portion that connects the substrate and the reinforcing plate to each other.

5. The optical sensor module according to claim 4,
wherein the connection portion connects the substrate and the reinforcing plate to each other with solder.

6. The optical sensor module according to claim 4,
wherein in a plan view viewed from a side facing the target object,
a plurality of connection portions each of which is formed of the connection portion are so provided as to surround the light emitter and the light receiver.

7. The optical sensor module according to claim 4,
wherein the connection portion is disposed in a region along a first edge of the substrate and a region along a second edge of the substrate that faces the first edge.

8. The optical sensor module according to claim 1,
wherein in a plan view viewed from a side facing the target object,
the reinforcing plate is so provided as to contain the light emitter and the light receiver.

9. The optical sensor module according to claim 1,
wherein in a plan view viewed from a side facing the target object,
the reinforcing plate has at least one hole section that exposes the light emitter and the light receiver.

10. The optical sensor module according to claim 9,
wherein the reinforcing plate has, as the at least one hole section, a first hole section that exposes the light emitter and a second hole section that exposes the light receiver.

11. The optical sensor module according to claim 2,
wherein in a plan view viewed from a side facing the target object,
the reinforcing plate has a first hole section that exposes the light emitter and a second hole section that exposes the light receiver, and
the light blocker is provided at least in a position between the first hole section and the second hole section.

12. The optical sensor module according to claim 1,
further comprising a detector at least including an amplification section that amplifies a detection signal from the light receiver,
wherein the detector is provided on the substrate.

13. The optical sensor module according to claim 12,
wherein the substrate is provided with a connector section electrically connected to a second substrate provided with a processing section that carries out a process based on the detection signal from the light receiver.

14. The optical sensor module according to claim 13,
wherein L1<L2 and L1<L3 are satisfied,
where L1 represents a distance from the connector section to the detector, L2 represents a distance from the connector section to the light emitter, and L3 represents a distance from the connector section to the light receiver.

15. The optical sensor module according to claim 1,
wherein the reinforcing plate is formed of a metal member or a resin member.

16. The optical sensor module according to claim 1,
wherein the deformable substrate is a flexible printed circuit.

17. A biological information detecting apparatus comprising the optical sensor module according to claim 1.

18. A biological information detecting apparatus comprising the optical sensor module according to claim 2.

19. An electronic instrument comprising the optical sensor module according to claim 1.

20. An electronic instrument comprising the optical sensor module according to claim 2.
